(19) United States
(12) Patent Application Publication
(10) Pub. No.: US 2011/0103655 A1
(43) Pub. Date: May 5, 2011

(54) FUNDUS INFORMATION PROCESSING APPARATUS AND FUNDUS INFORMATION PROCESSING METHOD

(76) Inventors: Warren G. Young, San Diego, CA (US); Masahiko Kobayashi, Nukata (JP); Yasuhiro Hoshikawa, Toyokawa (JP)

(21) Appl. No.: 12/611,439

(22) Filed: Nov. 3, 2009

(51) Int. Cl. G06K 9/00; A61B 3/12

(52) U.S. Cl. 382/128; 351/206

(57) ABSTRACT

The present inventions relates to a fundus imaging device integrated with a computer processing to comprise a fundus information processing apparatus and method for executing the same in order to produce a panoramic fundus image and to obtain blood vessel information of the fundus. The apparatus and method processes a first fundus image and a second fundus image acquired with a fundus imaging device. The fundus information processing apparatus and method extracts blood vessel shapes from the first and second fundus images, and identifies branching points of the blood vessel of the blood vessel shape through image processing. Further, a plurality of line segments are obtained by interconnecting two predetermined branching points and thereafter identification of two common line segments of the first and second fundus images. The apparatus and method determines relative positional relationship between the branching points constituting at least two of the line segments and thereafter performs comparison operation followed by an alignment operation. By the alignment a panoramic image of the plurality of fundus images is produced along with blood vessel information of the fundus.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates generally to an apparatus and method of biological tissue imaging, including the imaging of the fundus of the eye in the field of ophthalmology. More particularly, the present invention relates to a fundus information processing apparatus that processes a fundus image including fundus information, especially blood vessel information, acquired with a fundus image pickup device, such as a fundus camera, and a fundus information processing program and method for use in such an apparatus.

Conventionally, a technique has been known that picks up a plurality of fundus images of an examinee by use of a fundus image pickup device such as a fundus camera, and overlaps (matches) these fundus images to obtain a panoramic image, thereby grasping a condition of the fundus of the examinee as demonstrated, for example, in U.S. Pat. No. 6,082,859 (corresponding to PCT Publication WO99/13763) issued to Okashita et al, the entire substance of which is incorporated herein by reference. According to such a technique, the examinee is guided to fix his/her eyes by using a fixation lamp fitted to the fundus image pickup device, thus obtaining a plurality of fundus images having different image pickup positions. In this case, position information of the fundus images corresponding to the positions of the fixation lamp are obtained and utilized when overlapping the plurality of fundus images. According to the technique of U.S. Pat. No. 6,082,859, positional relationships of the plurality of fundus images are set up by using the position information of the fixation lamp, thus overlapping these fundus images.

Further, to improve the resolution of a panoramic image, such techniques may possibly involve using fixation lamp information to roughly align fundus images and then overlapping these images manually or automatically. Such overlapping may utilize, among other things, blood vessel information. For example, in the case of manual overlapping, after rough alignment, an operator overlaps two fundus images in such a manner that their points of identity, such as blood vessel shapes, may align and overlap in these fundus images. In the case of automatic overlapping also, after rough alignment, image processing is performed on the boundaries of two fundus images, to overlap these fundus images in such a manner that their blood vessel shapes, used as points of identity, may align and overlap.

Another technique is known that acquires a blood vessel shape, such as branching information, which serves as blood vessel information from a fundus image. The obtained information is used when diagnosing a condition of examinee's eyes (see Japanese Patent Application Laid-Open Publication (JP-A) No. 7-178056, for example). According to such a technique, image processing is initiated from information of a feature in the fundus. In the case of Japanese Patent Application Laid-Open Publication (JP-A) No. 7-178056, imaging is initiated from the position of the optic papilla in order to obtain shapes of the blood vessels contained in the fundus image.

However, the fundus image matching technique disclosed in U.S. Pat. No. 6,082,859 requires fixation lamp information pieces that correspond to the respective fundus images and, therefore, it is difficult to overlap the plurality of fundus images if they are all acquired with a fundus image pickup device. Further, the overlapping process, no matter whether manual or by means of image processing, may take an extremely long lapse of time if the positions of the plurality of fundus images are unknown.

Also, the technique disclosed in Japanese Patent Application Laid-Open Publication (JP-A) No. 7-178056 requires knowing a position of the optic papilla in order to simplify image processing. Therefore, it is necessary to input or identify the position of the optic papilla manually or automatically, otherwise, it is extremely difficult to obtain blood vessel information in a fundus image without identifying the location of the optic papilla.

As such, there is a need in the art for obtaining and aligning multiple fundus images to generate a valid and useful panoramic fundus image without the use of a fixation lamp and/or corresponding each component image to the location of a fixation lamp. In addition, there is the need in the art for obtaining and aligning multiple fundus images to generate a valid and useful panoramic fundus image without requirement of locating and inputting the optic papilla or other fixed feature of the eye, in order to generate the fundus image.

### BRIEF SUMMARY

The present invention overcomes shortfalls of the conventional techniques by providing a fundus information processing apparatus and method capable of efficiently and quickly matching and aligning a plurality of fundus images based on blood vessel information without using fixation lamp information. In addition, the present invention provides a fundus information processing apparatus and method capable of acquiring blood vessel information across a plurality of fundus images based on blood vessel information of at least two of these fundus images without using the information of the optic papilla.

In an aspect of the invention, a fundus information processing apparatus processes a first fundus image and a second fundus image acquired with a fundus image pickup device and acquires at least one of a panoramic fundus image and fundus blood vessel information. The fundus information processing apparatus includes a blood vessel extraction unit, a line segment information acquisition unit, a line segment identification unit, a branching point information calculation unit, a comparison operation unit, and an alignment processing unit. The blood vessel extraction unit extracts blood vessel shapes from the first and second fundus images, and extracts branching points of the blood vessel of the blood vessel shape through image processing. The line segment information acquisition unit acquires, in each of the first and second fundus images, information of a plurality of line segments obtained by interconnecting two predetermined branching points by using the plurality of branching points extracted by the blood vessel extraction unit. The segment identification unit identifies at least two of the line segments common to the first and second fundus images by using the line segment information obtained by the line segment information acquisition unit. The branching point information calculation unit calculates, in each of the first and second fundus images, branching point information that indicates a relative positional relationship between the branching points constituting at least two of the line segments identified by the line segment identification unit. The comparison operation unit performs comparison operation on first branching point information of the first fundus image and second branching point information of the second fundus image which are calculated by the branching point information calculation unit. The alignment processing unit performs alignment processing on the first and second fundus images based on a result of the comparison operation by the comparison operation unit.

In another aspect of the invention, a fundus information processing program is executed by an arithmetic unit of a computer to process a first fundus image and a second fundus image acquired with a fundus image pickup device, and acquire at least one of a panoramic fundus image and fundus blood vessel information. The fundus information processing program includes a blood vessel extraction step, a line segment information acquisition step, a line segment identification step, a branching point information calculation step, a comparison operation step, and an alignment processing step. The blood vessel extraction step extracts blood vessel shapes from the first and second fundus images, and extracts branching points of the blood vessel of the blood vessel shape through image processing. The line segment information acquisition step acquires, in each of the first and second fundus images, information of a plurality of line segments obtained by interconnecting two predetermined branching points by using the plurality of branching points extracted at the blood vessel extraction step. The line segment identification step identifies at least two of the line segments common to the first and second fundus images by using the line segment information obtained at the line segment information acquisition step. The branching point information calculation step calculates, in each of the first and second fundus images, branching point information that indicates a relative positional relationship between the branching points constituting at least two of the line segments identified at the line segment identification step. The comparison operation step performs comparison operation on first branching point information of the first fundus image and second branching point information of the second fundus image which are calculated at the branching point information calculation step. The alignment processing step performs alignment processing on the first and second fundus images based on a result of the comparison operation at the comparison operation step.

According to the present invention, it is possible to match a plurality of fundus images based on blood vessel information without using fixation lamp information. It is also possible to acquire blood vessel information across a plurality of fundus images based on blood vessel information of at least two of these fundus images without using the information of the optic papilla.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is an illustration showing the constitution of a fundus information processing apparatus according to an embodiment of the present invention;

FIG. 2 is an explanatory diagram of gridding processing of the present invention;

FIG. 3A are schematic diagrams showing two fundus images having different photographing regions except in the same examinee's eye in which some of the regions are common to them;

FIG. 3B are two branching diagrams in which blood vessel branching points are extracted from the respective two fundus images shown in FIG. 3A;

FIG. 4 is a schematic diagram showing a state where a combination of the branching points that form a line segment common to the two branching diagrams is extracted;

FIG. 5 is a diagram showing a panoramic fundus image generated by overlapping the two fundus images;

FIGS. 6A and 6B are explanatory schematic diagrams of boundary processing; and

FIG. 7 is a flowchart showing a series of steps of a method of the present embodiment.

### DETAILED DESCRIPTION

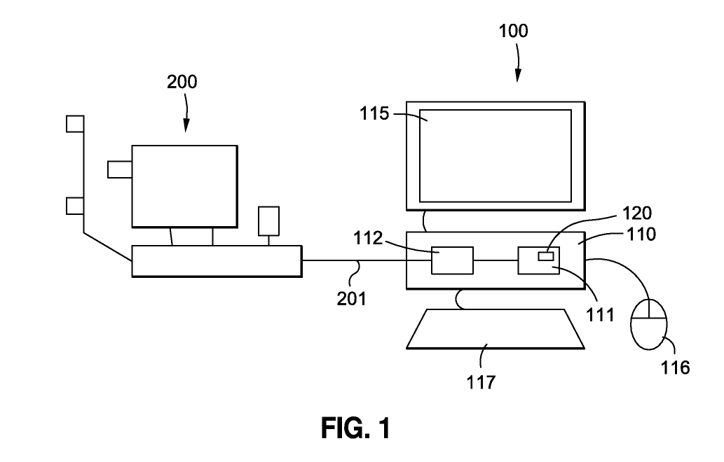

Embodiments of the present invention will be described with reference to the drawings. Referring particularly to FIG. 1, an illustration is provided showing the structure and organization of a fundus information processing apparatus according to an embodiment of the present invention.

The fundus information processing apparatus 100 is connected to a fundus camera 200, which serves as a fundus image pickup device that photographs the fundus of the eye of an examinee. It is to be noted that the fundus information processing apparatus 100 and the fundus camera 200 may be in electrical communication with each other directly with a cable 201 or other wired means, or indirectly where data can be transferred between them through a network or other computer system. In addition, it is contemplated by the present invention that that the camera 200 could also be in wireless or optical communication with the apparatus 100. It is additionally contemplated that in the present invention that data from the camera 200 could be loaded onto a memory card which is manually transported to an interface of the apparatus 100 and uploaded for further processing. The fundus camera 200 comprises an illumination optical system that illuminates the examinee's fundus, an image pickup optical system that photographs the illuminated examinee's fundus, an observation optical system that observes the examinee's fundus for the purpose of alignment, etc. The fundus camera 200 picks up a color fundus image by irradiating the fundus with visible flash light or pick up a fluorescent fundus image of the examinee by irradiating with exciting light the contrast-enhanced fundus blood vessels of the examinee dosed with a fluorescent agent. Those fundus images are subjected to digital processing to give electronic fundus images. Since the fundus camera 200 picks up an image of the examinee's fundus directly, the picked-up fundus image may contain a wide range of the fundus blood vessels of the examinee. The present invention contemplates the use of other fundus image pickup devices, including, but not limited to, a scanning laser eye speculum and a digital slit lamp (slit lamp equipped with a digital camera).

The fundus information processing apparatus 100 comprises a personal computer (PC), whose PC body 110 includes a memory 111, such as a hard disk which serves as data storage to store examinee's identification information, the fundus images and data related to the images such as the date the image was taken, etc. The fundus imaging process apparatus further includes a central processing unit (CPU), which will hereinafter be referred to as “arithmetic-and-control section” 112 which serves as an calculation unit that processes the fundus information and related information of the examinee. The PC body 110 includes connections to a color monitor 115 which serves as a display unit and a mouse 116 as well as a keyboard 117 which serve as an input or peripheral unit. It is to be noted that the arithmetic-and-control section 112 plays the role of performing predetermined image processing or image analysis on an acquired fundus image.

In an embodiment of the present invention, the fundus information processing apparatus 100 and the fundus camera 200 are connected to each other with a cable 201. As described above, a fundus image obtained by the fundus camera 200 is written into the memory 111 when the arithmetic-and-control section 112 receives an instruction to input the fundus image. In this case, photographing information (for example, examinee's identification information and eye information, photographing date, etc.) accompanying the fundus image is also stored.

The present embodiment provides a means to align and then overlap a plurality of fundus images of the examinee's eye picked up with the fundus camera 200 by using the fundus information processing apparatus 100 to thereby obtain a panoramic image and also obtain blood vessel information (blood vessel shapes in this case) of the fundus across the plurality of fundus images. It is to be noted that the above plurality of fundus images refer to fundus images of the same examinee which have photographing regions partially overlapping each other. Specifically, a fundus information processing program 120 stored in the memory 111 is executed by the arithmetic-and-control section 112, which consecutively processes a plurality of fundus images stored in the memory 111, to generate a panoramic image, thereby extracting and/or assembling blood vessel information. The arithmetic-and-control section 112 provides an execution unit that executes processing steps (for example, overlapping step etc.) described below.

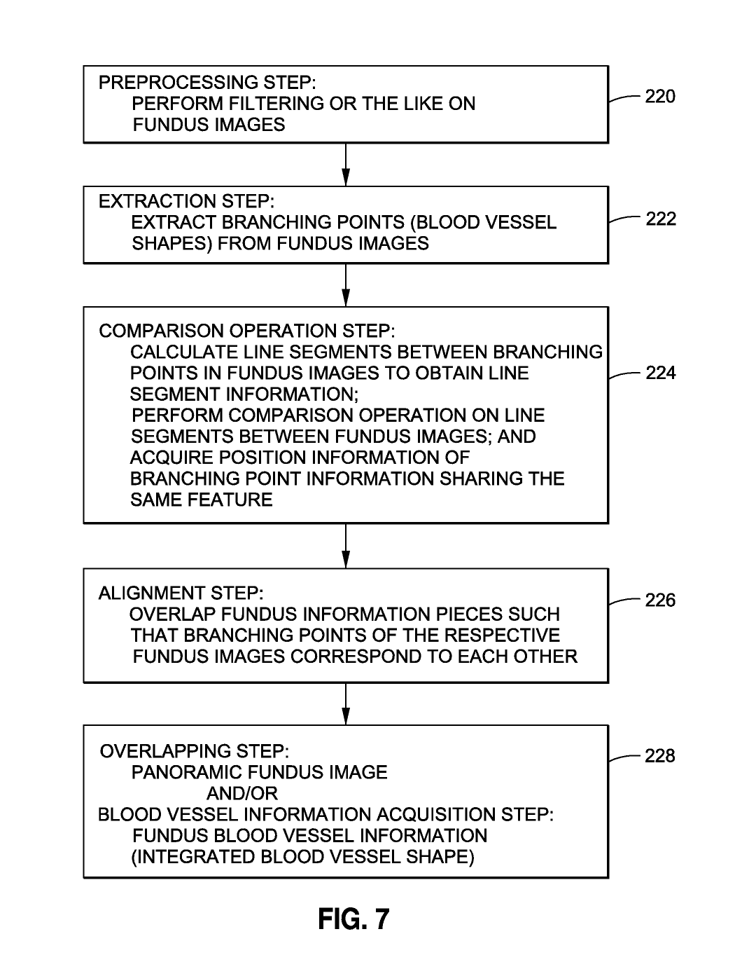

In FIG. 7A, there is shown a flowchart demonstrating a series of steps of a processing procedure and method which is carried out by executing the program 120. The processing is roughly divided into the following steps: a preprocessing step 220 of performing filtering etc. of fundus images; an extraction step 222 of extracting fundus information (blood vessel shapes) from the fundus images; a comparison operation step 224 of performing comparison operation on the fundus images based on the extracted blood vessel shapes, and an alignment step 226 of aligning the images by matching portions of the blood vessel shapes common to the fundus images. Finally an overlapping step and/or a blood vessel information acquisition step 228 is provided to produce the panoramic fundus image when completing the overlapping step or acquiring blood vessel information when the blood vessel information acquisition step is performed.

The fundus images are subjected to image processing referred to as preprocessing step 220 before extracting the fundus information in the extraction step 222. The preprocessing includes, but is not limited to, lens distortion correction, sub-sample processing, mask processing, level correction, and smoothing filtering.

The lens distortion correction, which refers to processing to reduce optical distortion which occurs along the peripheries of a fundus image, is performed to correct image distortion due to the image pickup optical system of the fundus camera 200 and the visibility of the examinee's eyes. If the distortion of the fundus images at their peripheries is corrected, the fundus images are better suited for overlapping when generating a panoramic image, which is described later. The distortion is corrected by using the optical design information of the fundus camera 200 and the visibility of the examinee's eyes as functions.

The sub-sample processing refers to processing to reduce the size of a fundus image. In the present embodiment, the image size (file size) is scaled down to ¼. With this, the quantities of image processing of the following stages, comparison operations, etc. have been reduced to smooth the overall processing. It is to be noted that the image size may be reduced to such an extent as not to degrade the features of the blood vessel shape. Therefore, the degree of degradation, if any, of the minute blood vessel etc. caused by the sub-sample processing must only be such as not to influence the features of the shape of the blood vessel of the overall fundus image. In this regard, it is contemplated by the present invention that the scaling down of the image size may be some other factor than ¼, however, any scaling is suitable that does not degrade or influence the features of the shape of the blood vessels in the fundus image.

The mask processing refers to processing to remove a circular mask peculiar to a fundus image acquired with the fundus camera etc. Pixels outside a predetermined mask are to be preset so as to be ignored in the later-stage processing. With this processing, the pixels to undergo operations are reduced to decrease the quantity of calculations, thereby smoothing the overall processing. It is to be noted that the circular mask is specific to the image pickup device such as a fundus camera, so that mask processing may not need to be performed on a fundus image acquired with a different type of the fundus image pickup device, for example, a scanning laser eye speculum.

The level correction refers to histogram processing to stretch the histogram of pixel information of a fundus image. This processing improves the contrast of the blood vessels to facilitate extracting of the blood vessel shapes in the later-stage processing. In the present embodiment, the processing may be performed to emphasize red colors in a color distribution of red, blue, and green of a fundus image. It is to be noted that level correction is not limited to a color-distribution image such as a color fundus image but may only need to improve the contrast of a black-and-white fundus image such as a fluorescent fundus image. Further, it is contemplated by the present invention that other colors may be emphasized when using epiluminescent markers, or in dealing with other types of tissues of differing body parts or differing animals under study.

The smoothing filtering refers to filtering a fundus image with a Gaussian filter, which is a smoothing filter. This reduces noise contained in a fundus image. This processing reduces the contrast of noise and minute images, for example, block noise etc. of a fundus image. The contrast may be decreased also of minute hemorrhage, exudation, etc. from the fundus. This processing improves a signal-to-noise (S/N) ratio in the image, thus facilitating the later-stage processing of extracting the blood vessel shapes. It is to be noted that the filter to be used is not limited to a Gaussian filter but may be any type as far as it is capable of reducing the noise in images. A median filter etc. may be used. The fundus images thus subjected to the series of preprocessing pieces are stored in the memory 111.

Next, the fundus images undergo extraction processing step 222 to extract blood vessel shapes, which are blood vessel information of these fundus images. In the present embodiment, pixel analysis is performed on the fundus images that have undergone the preprocessing, to analyze a difference in luminance between the fundus and the blood vessels, thereby extracting a shape of the blood vessels. The pixel analysis to be utilized may be a feature extracting technique (for example, edge detection) by use of the conventional image processing.

It is to be noted that in the present embodiment a seed point, which provides a stepping stone to extraction of the blood vessel shape, is utilized in order to extract the blood vessel shapes efficiently. To set up the seed point, processing referred to as gridding processing is carried out. The blood vessel shape is traced starting from a seed point obtained in the gridding processing, thus enabling the blood vessel shape to be speedily extracted.

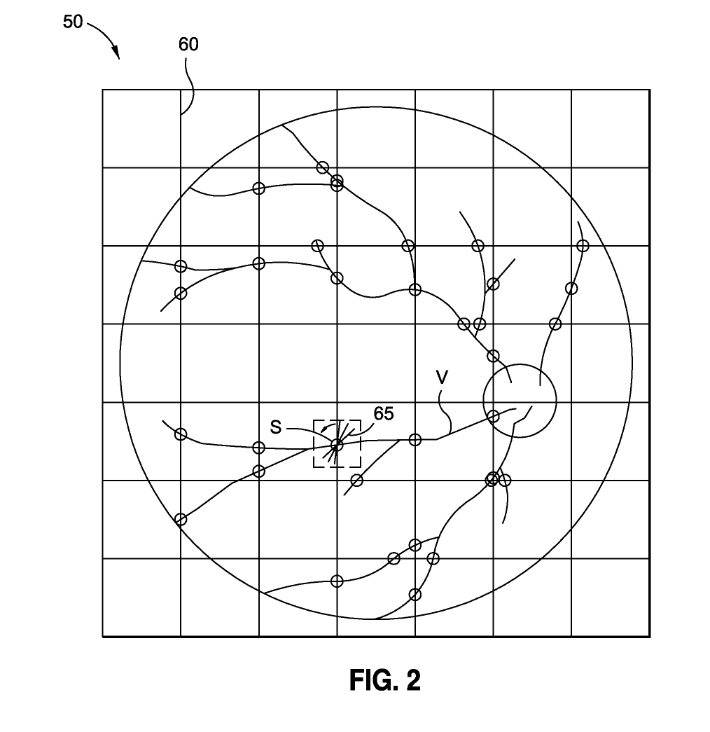

FIG. 2 is an explanatory diagram of the gridding processing. In gridding processing, first, a plurality of lines are set up in a mesh-shape on a fundus image to be processed. In the present embodiment, the plurality of lines are set up in a mesh shape (lattice shape in this case) formed so as to cut across the fundus blood vessels. It is to be noted that for ease of explanation, a grid 60 formed of a seven-by-seven square lattice is, in this case, overlapped with the entire regions of a fundus image 50.

By using only red components of the fundus image 50, the blood vessels are extracted which intersect with (run over) the lines of the grid 60. Pixel distributions on the lines of the grid 60 are compared to each other so that a portion of the line having an enhanced red component as compared to a background (retina R) is given as the blood vessel V. In this case, the gradient of the luminance on the line is calculated thereby to determine a width (outline) of the blood vessel V on the line and also determine the center of the blood vessel V. Specifically, the blood vessel is scanned starting from its internal point having a low luminance value toward its outside to search for a position (retina) where the luminance increases. The thus encountered boundary may provide the blood vessel wall. The middle point of a line segment interconnecting both-side boundaries thus extracted is set up as a seed point S, whose position is then stored in the memory 111. A gravity point may be calculated from the luminance distribution of the blood vessel and set up as the seed point. The middle point (center point) between both-side blood vessel walls may be set up as the seed point. Such a seed point S is set up on all the blood vessels that intersect with any of the lines. The seed point S may be extracted from a gray-scaled fundus image.

It is to be noted that the grid is not limited to a lattice in shape but may be of any shape as far as lines and the blood vessels are set to as to intersect with each other at a predetermined pitch. For example, a triangular lattice shape or a honeycomb shape may be used.

Next, the shape of the blood vessel V is searched for by using each of the seed points S thus set up. In this case, the fundus image 50 is processed in gray scale display. Now, a seed point S enclosed by a dotted line in the figure is noticed. Line scanning is performed in all directions around the seed point S as an axis (rotation axis). In the line scanning, a line 65 is set up which is about long enough to capture the running blood vessels. In the present embodiment, the line 65 used in line scanning is set up to have a length of 10 pixels in the back-and-forth direction around the center of the seed point S. The lines 65 are scanned for each sampling angle of such a magnitude as to be able to capture the blood vessels. In this case, the lines are scanned for each 20 degrees.

As aforesaid, the blood vessel V is extracted from a luminance distribution on the lines 65 and traced in either a forward running direction (toward the tip) or a backward direction (toward a base end where the optical papilla exists). In this case, it is traced in a descending direction of the luminance value. By doing such processing, a running direction (directivity) of the blood vessel with respect to the seed point can be estimated.

Next, to efficiently trace the extracted blood vessel V in the running direction, line scanning is performed by setting up a range containing the running direction of the blood vessel V. Specifically, a line passing through the seed point S and the current point extracted as the blood vessel V is calculated, along which line the line scanning is performed around the current point at ±40 degrees with respect to the tracing direction.

In the above trace, a description will be given of an extracting method in a case where branching points or intersection points of the blood vessel V are scanned. A branching point refers to a position where the blood vessel branches off, that is, a position where one blood vessel splits into two branching blood vessels. Therefore, the blood vessel may extend in three directions as viewed from the branching point. On the other hand, an intersecting point refers to a point where one blood vessel and another blood vessel (for example, artery and vein) intersect with each other, that is, a point where they are observed in a condition where they overlap each other when the fundus is photographed squarely. Therefore, the blood vessel extends in four directions as viewed from the intersecting point. A branching point and an intersecting point may be distinguished from each other depending on whether the blood vessels going out of a certain point are odd-numbered or even-numbered.

From these, if scanning from a certain point comes up with the two blood vessels, this point is judged to be a branching point B; and if it comes up with the three blood vessels, this point is judged to be an intersecting point C (not shown). Then, the positions (coordinates) of the branching point B and the intersecting point C are stored in the memory 111.

Such processing is performed on each of the seed points S until all the blood vessels V in the fundus image 50 may be extracted through tracing. The shapes (consecutive coordinate positions) of the blood vessels V are stored in the memory 111. It is to be noted that a location once traced as the blood vessel V is never to be traced again because its information is stored. This prevents the efficiency of the processing from being lowered.

By thus extracting the blood vessel shapes, the following advantages will be discussed. As compared to a method for searching a fundus image at random to trace the blood vessel by predetermining an initial position, the method of the present embodiment has a high efficiency because a seed point is set up as an initial point for tracing. The efficiency may be improved further because such a seed point exists on the few blood vessels. Further, as compared to the method for predetermining the optic papilla etc. as a feature point and setting it up as an initial position and then tracing the blood vessel starting from there toward its outside (toward end side), the method of the present embodiment can extract the blood vessel shapes speedily because it can omit the step of extracting the optic papilla. Further, the blood vessel shapes can be extracted even in a fundus image containing no optic papilla.

It is thus possible to extract overall blood vessel shapes from a fundus image. Coordinates information of the blood vessel shapes, width information of the blood vessels, branching point information (branching position information) containing coordinate positions of branching points of the blood vessels, coordinates information of the blood vessel intersecting points, etc. are combined with identification information etc. of the fundus image and stored in the memory 111. The fundus images subjected to such extracting processing are each stored in the memory 111.

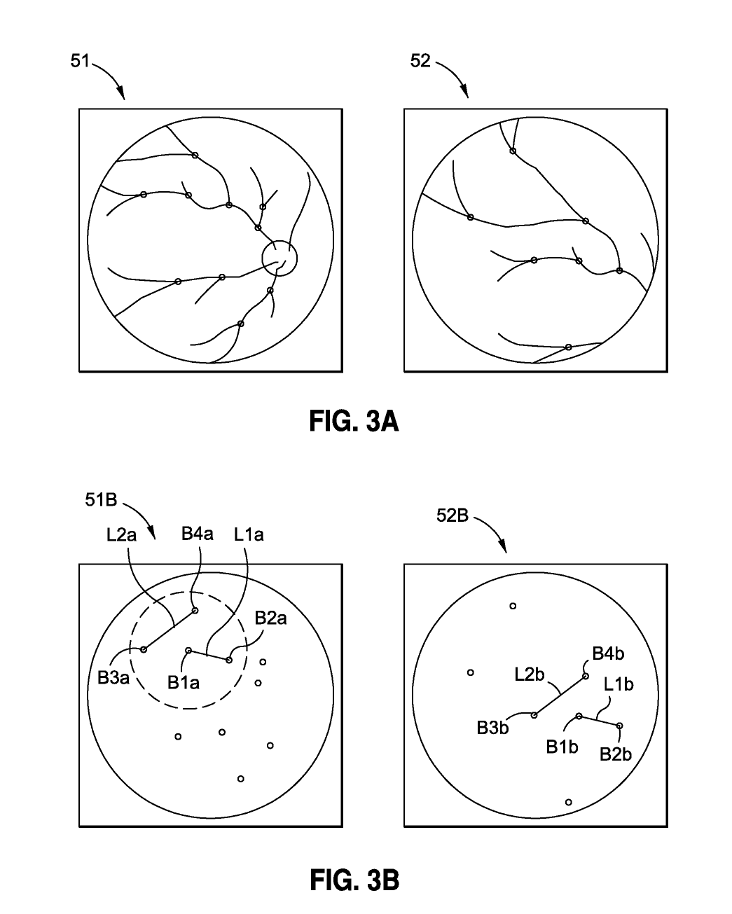

Next, a description will be given of processing to cross-check the blood vessel shapes of the different fundus images by using the branching point information contained in the blood vessel information and overlap the fundus images, thus obtaining a panoramic image. It is to be noted that the blood vessel information (for example, blood vessel shapes) of the fundus across all the fundus images required to obtain the panoramic image is also obtained by performing the aforesaid processing. FIG. 3A show fundus images having different photographing regions on the same examinee's eye and are schematic diagrams showing two fundus images 51 and 52 having a partially common photographing region. On the other hand, FIG. 3B are branching diagrams 51B and 52B in which blood vessel branching points are extracted from the respective fundus images 51 and 52 shown in FIG. 3A. With respect to the fundus image 51 serving as a first fundus image, the fundus image 52 serving as a second fundus image is of the same examinee's eye photographed at a position different from that of the fundus image 51. The branching diagrams are in fact managed in the memory 111 as numerals having coordinates.

Subsequently, matching processing (alignment processing) 226 is performed on the original fundus images based on relationships between the branching points of the branching diagrams 51B and 52B. Matching processing of the present embodiment is roughly divided into the following steps.

Two branching points are picked up from among those in the branching diagram and interconnected to form a line segment, whose line segment information is then obtained such as its length, angle, etc. Such line segment information is obtained of all the branching points. Alternatively, line segment information is obtained of branching points having a predetermined relationship. Such line segment information is calculated for each of the branching diagrams and compared to that of the different branching diagram, thus obtaining a common line segment. Further, for each branching diagram, branching point information is obtained which indicates a relative positional relationship between branching points that constitute the obtained common line segment. Comparison operation is performed on the branching point information pieces of the respective branching diagrams, so that based on an obtained result of the comparison operation, matching processing is performed on the different fundus images.

Next, a specific example about the matching processing will be described. In the first step, features of the fundus images 51 and 52 are extracted from their information. In this case, line segments interconnecting branching points are calculated using the branching diagrams 51B and 52B. One branching point B1 is noticed in the branching diagram, to set up a range around this branching point B1 in which other branching points are to be searched for. This range may be set up beforehand and only needs to contain at least one other branching point but not so many. In the present embodiment, around the branching point B1 as a center, a circle (dotted line in the figure) is set up whose radius is roughly a half the diameter of the fundus image. In this range, the branching point B1 forms a line segment to reach another branching point. Further, also for a branching point present in a predetermined range around the branching point B1 and an another branching point outside the range, a line segment is formed which reaches the different branching point in the predetermined range in the same way. The line segment information such as a length of the formed line segment and an angle formed by the line segment with respect to a baseline (for example, X-axis) is stored in the memory 111. In such a manner, the line segments calculated for the branching diagrams 51B and 52B respectively are all listed and their line segment information is stored in the memory 111 (line segment information is acquired for each of the branching diagrams).

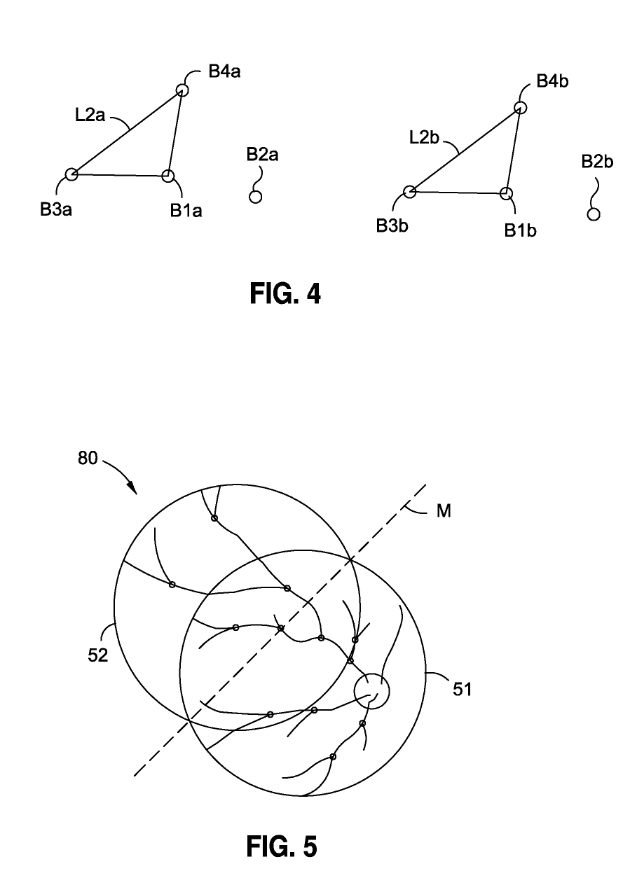

In the next second step, based on the line segment information obtained in the first step, common line segments (branching points) in the branching diagrams 51B and 52B are extracted and subjected to processing (line segment identification processing) of deciding whether they are a location (which has the same feature) of the same blood vessel shape. At least two common line segments in the branching diagrams are noticed. In this case, it is assumed that the line segment information of a line segment L1a formed by branching points B1a and B2a in the branching diagram 51B is common to the line segment information of a line segment L1b formed by branching points B1b and B2b in the branching diagram 52B and that the line segment information of a line segment L2a formed by branching points B3a and B4a in the branching diagram 51B is common to the line segment information of a line segment L2b formed by branching points B3b and B4b in the branching diagram 52B. FIG. 4 shows a state in which a combination of branching points that form common line segments in the branching diagrams 51B and 52B is picked up. Thus, the common line segments in the branching diagrams 51B and 52B highly possibly represent the same blood vessel shape (location). Therefore, by obtaining the relative positional relationship between the branching points of common line segments in each of the branching diagrams, the line segments are decided on whether they represent the same blood vessel shape.

There are four branching points (B1a to B4a) that form the common line segments L1a and L2a extracted in the branching diagram 51B. In order to grasp the positional relationships among those branching points B1a to B4a, at least three of those branching points are used to form a graphic. In the present embodiment, the three branching points B1a, B3a, and B4a have been used to form a triangle. Similarly, in order to grasp the positional relationships among the branching points B1b to B4b that form the common line segments L1b and L2b extracted in the branching diagram 52B, at least three branching points are used to form a graphic. In this case, three such branching points are selected as to have the same relationship as that of the branching points selected in the branching diagram 51B. The arithmetic-and-control section 112 calculates branching point information for each of the branching diagrams. It may come in the first branching point information in the case of the branching diagram 51B and the second branching point information in the case of the branching diagram 52B. In this case, information of each of the formed triangles is obtained (for example, internal angle, contour length, length of each side, etc.). Then, the arithmetic-and-control section 112 compares the first branching point information and the second branching point information to each other. In the present embodiment, it respectively compares line segment length of a triangle formed by a line segment (first side) and one point on a different line segment, the length of a line segment (second side) formed by one point on a line segment different from this line segment, and the angle formed by the two line segments (first and second sides). In other words, the arithmetic-and-control section 112 compares the information (shape) of a triangle formed by the line segment L2a and the point B1a and the information (shape) of a triangle formed by the line segment L2b and the point B1b. In addition to these triangle information pieces, a contour length of the triangle may be used.

As a result of comparison by the arithmetic-and-control section 112, if the information pieces of the two triangles agree, the triangles can be considered to be identical, the common line segments (branching points) in the branching diagrams 51B and 52B are judged to indicate a common blood vessel shape (same site). On the other hand, if those two triangles do not agree, common line segments indicate different sites. The comparison results are stored in the memory 111. It is to be noted that the expression of “agree” as referred to here does not mean perfect coincidence but may have a predetermined allowable range in which they can be judged to agree. Such an allowable range only needs to allow a change in shape of triangles that may be caused by photographing conditions and a degree of accuracy in image processing.

Further, in comparison operation of the triangles, the arithmetic-and-control section 112 calculates a shift amount (moving distance) of triangles judged to agree. The shift amount as referred to here indicates the amount of a relative displacement between the compared triangles which is required to overlap these triangles with each other. The shift amount refers to information (target information) required in the later-described alignment of fundus images, and indicates relative positions of two fundus images aligned with each other. In the present embodiment, the shift amount is obtained by conducting affine transformation on all the points of triangles to be paired with each other. It is to be noted that the shift amount of the triangles to be paired may be calculated with reference to the gravities or specific points and sides of the respective triangles. The shift amount is calculated for each pair of triangles (paired triangles) judged to agree in each of the branching diagrams 51B and 52B and stored in the memory 111. Although the example has been described in which one paired triangle is present in each branching diagram, actually there may be cases where a plurality of triangles are formed in each of the branching diagrams and, correspondingly, the number of pairs is more than one. Ideally, the shift amount required to align the branching diagrams (fundus images) with each other should be the same in any selected one of the pairs but, actually, changes somewhat depending on, for example, distortion in the picked-up images and the accuracy in image processing. Therefore, if the number of the pairs is more than one, the shift amount is obtained for each of the pairs and stored in the memory 111 in a condition where it is compared to the information of the compared triangles paired with each other.

It is to be noted that such comparison is performed on at least two common line segments. The more line segments are compared, the more precisely a blood vessel shape common to the two fundus images can be obtained. Although the present embodiment obtains the positional relationship between branching points by using a triangular shape, the present invention is not limited thereto. Use of the method of the present invention only needs to be capable of obtaining by operations a positional relationship of branching points to be compared. For example, two line segments may be utilized to use a rectangle or at least three common line segments may be used at a time to form a triangle or any other polygon by using branching points that form those line segments. Further, at least three common line segments may be extracted to compare graphics formed by branching points that constitute each of the line segments.

Further, although the present embodiment has compared two fundus images, the present invention is not limited thereto; three or more fundus images can be compared to each other. If a number of fundus images are compared, one such of the fundus images as to contain the largest number of sites common to them provides a central fundus image.

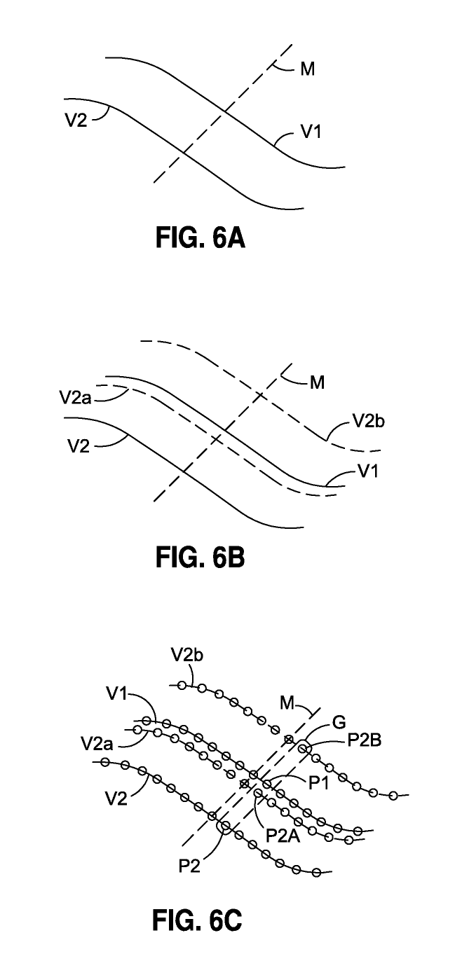

After the relationships between the branching diagrams are thus extracted, those information pieces are used to perform alignment processing on the fundus images, which is followed by boundary processing and overlapping processing in this order. FIG. 5 is a diagram showing a panoramic fundus image 80 generated by overlapping the fundus images 51 and 52. FIGS. 6A and 6B are explanatory schematic diagrams of the boundary processing.

For example, if fundus images are aligned to then undergo overlapping processing based on those relationships between the branching diagrams, distortion etc. at the peripheries of the fundus images may give rise to undesirable blood vessel linkage between the fundus images. To display such blood vessel linkage between the fundus images as natural as possible, boundary processing is performed in the present embodiment. It is to be noted that the following description is based on the assumption that there are three combinations of triangles to be paired with each other and the three different shift amounts have been calculated for each of the pairs.

The arithmetic-and-control section 112 overlaps the fundus images 51 and 52 with each other using shift amounts calculated on the basis of a certain one of the pairs, and sets up a boundary region at a location where two fundus images overlap each other. The present embodiment assumes an intermediate line M that passes points where peripheral circles of the fundus images 51 and 52 agree respectively (see FIG. 5). A region as large as several tens to several hundreds of pixels is set up symmetrically with respect to the intermediate line M. This example takes notice of the fundus image 51 side blood vessel V1 that run across the intermediate line M and the fundus image 52 side blood vessel V2 that should be overlapped with the blood vessel V1. It is to be noted that curves (V1, V2) in the figure indicate only the intermediate lines of the blood vessels. Since the picked-up images have distortion and some differences in magnification, it is difficult to completely overlap (link) the common blood vessels with each other even by overlapping the fundus images 51 and 52 based on the shift amounts, thus resulting in a displacement in the blood vessels V1 and V2 which should overlap with each other, as shown in FIG. 6A. To solve this problem, a plurality of pieces of the shift amount information stored in the memory 111 are each applied to determine shift amounts that provide such conditions that the blood vessels in this region may seem to be linking with each other smoothly. As shown in FIG. 6B, the fundus images 51 and 52 are shifted with respect to respective shift amounts at a position (in the boundary region) around the intermediate line M, thus tracing a blood vessel shape starting from the blood vessel V1. It is to be noted that although the intermediate line M is assumed for each of the shift amounts, for ease of explanation, here, a shift in position of the blood vessels (V2, V2a, V2b) caused by the respective shift amounts is assumed to be of parallel displacement along the intermediate line M. It is to be noted that in FIG. 6B, the blood vessels V2a and V2b are indicated by a dotted line, which have been drawn respectively based on shift amounts different from that of the blood vessel V2. The arithmetic-and-control section 112 calculates a relevance ratio between the two blood vessels (the blood vessels in the fundus images 51 and 52) in accordance with the respective shift amounts. The arithmetic-and-control section 112 decides that such a shift amount as to correspond to the highest relevance ratio provides a condition under which the blood vessels may link most smoothly in this boundary region. Based on the shift amount with the highest relevance ratio, overlapping on the fundus images is performed.

The relevance ratio in the present embodiment will be calculated by the arithmetic-and-control section 112 as follows. As shown in FIG. 6C, sampling is performed over points of the blood vessel at an interval between several pixels and several tens of pixels in the directions of the respective fundus images with respect to the intermediate line M. For example, sampling is performed on one point on the intermediate line M and the horizontal seven points with respect to the intermediate line M. The points separate from the intermediate line M by the same distance are classified into a group (for example, group G), thus calculating the distance at each of the points in the group. For example, the distance of each of points P2, P2a, and P2b corresponding to point P1 on the blood vessel V1 is calculated from the blood vessels V2, V2a, and V2b, respectively. If these distances satisfy a predetermined criterion with respect to point P1 (for example, distance of several pixels or less), the points are decided as matching. All the points in each group are decided on whether they match or not. The number of the points thus decided as matching is defined as a blood vessel-specific relevance ratio and stored in the memory 111. In the figure, it is decided that the blood vessel V2a matches the blood vessel V1 most. The blood vessel relevance ratio is thus determined in the boundary region. It is to be noted that if there are a plurality of the blood vessels running across the intermediate line M, such a shift amount is employed as to have the largest average value of the calculated relevance ratios of those blood vessels. Alternatively, such a shift amount may be used as to be of the blood vessel having the largest relevant ratio. It is to be noted that the blood vessel relevance ratio can be calculated by any method as far as it is capable of determining the distance between and the difference in shape of the blood vessels.

Such boundary processing reduces an influence of errors in the shift amount (shift amount of triangles here) of the graphics calculated from characteristics extracted in different branching diagrams, thus providing appropriate linkage between blood vessel shapes when forming a panoramic fundus image. By thus calculating a relevance ratio between the blood vessels running across fundus images from a plurality of shift amounts and performing overlapping processing on the fundus images based on a shift amount that gives a high relevance ratio value, it is possible to exclude a relationship of originally disagreeing site features that have been mistakenly judged to agree. Further, by utilizing the aforesaid triangle shift amounts calculated beforehand, it is possible to perform overlapping processing (aligning processing) efficiently while suppressing an amount of calculations.

The boundary processing may be to relatively move the target two blood vessels upward, downward, right, or left, or to relatively rotate the two blood vessels in a boundary region, as well as to move the two blood vessels parallel with respect to an intermediate line to calculate the relevance ratio. Further, although the present embodiment has employed a scheme of calculating a relevance ratio through comparison as shifting the two blood vessels based on the aforesaid triangle shift amounts, the present invention is not limited thereto. Such a scheme may be employed as to shift the two blood vessels by each predetermined pitch (for example, one pixel), thus calculating a relevance ratio between the two blood vessels.

Further, a location where the fundus images 51 and 52 overlap each other is subjected to blending processing as described below. The fundus images to be overlapped have masks removed therefrom to become circular in shape. Therefore, the circles overlap each other at a location to undergo blending processing. Blending processing is performed in accordance with a distance from the center of each of the fundus images 51 and 52. In this case, in the processing, luminance values of the overlapping pixels of the fundus images 51 and 52 are blended, to draw the image. In regions having a roughly equal distance from the center of each of the fundus images 51 and 52, the luminance values of the fundus images 51 and 52 are evenly blended (averaged), to draw the image. As the center of the fundus image 51 or 52 is approached from the region, a weight of luminance values to be blended is changed in drawing. Such blending processing may smooth a change in luminance around the boundaries between the fundus images 51 and 52, thereby providing natural view of the panoramic fundus image 80.

The fundus image overlapped through these processing pieces is stored as the panoramic fundus image 80 in the memory 111. It is to be noted that the fundus image to be overlapped may require relative scaling.

It is to be noted that as in the case of overlapping fundus images, processing is performed to calculate fundus blood vessel information in order to integrate the shape of the blood vessels across the fundus images 51 and 52. Based on alignment of branching points in different branching diagrams, the arithmetic-and-control section 112 matches (merges) blood vessel information (coordinate information, pixel information) pieces of a location common to the respective blood vessel shapes of the fundus images 51 and 52. In the present embodiment, the blood vessel information pieces of the common locations are averaged. In such a manner, the blood vessel shapes (blood vessel information pieces) across the fundus images 51 and 52 are integrated to acquire the fundus blood vessel information. The acquired fundus blood vessel information is stored in the memory 111.

The fundus blood vessel information thus acquired is managed as follows. If a plurality of fundus images that make up the fundus blood vessel information contain the optic papilla, the arithmetic-and-control section 112 determines the position of the papilla as follows. Image processing is performed to extract a region having a high luminance value and a large feature from a fundus image and calculate a luminance distribution in the region, thus extracting a periphery of the papilla. The arithmetic-and-control section 112 manages as a tree structure the blood vessel that extends from the papilla as a base toward the end (tip) thereof. In this case, the arithmetic-and-control section 112 extracts the number of the blood vessel branches, a branching shape of the blood vessel, a length of the blood vessel between branching points, and a width of the blood vessel from a shape of the blood vessel and manages them. The extracted information is stored in the memory 111.

It is thus possible to align a plurality of fundus images based on their blood vessel shapes (blood vessel information) without using fixation lamp information and, further, overlap those images, thus acquiring a panoramic fundus image having a high resolution. Further, it is possible to acquire fundus blood vessel information across a plurality of fundus images based on their blood vessel shapes independently of the information of the optic papilla. It is to be noted that the blood vessel shapes can be managed as a tree structure that has the branching point information of branching points starting from the optic papilla as the base up to the end thereof. Further, in addition to the tree structure, it is possible to manage also the blood vessel length, width, intra-blood vessel luminance distribution, etc. The blood vessel length between the branching points may come in a linear distance in an image or a distance based on a blood vessel shape. These information pieces can be used to screen the fundus diseases of the examinee's eyes, such as a systemic illness, etc.

Although the present embodiment has employed a scheme of performing boundary processing and then overlapping processing on the fundus images and blood vessel information acquisition processing, the present invention is not limited thereto. Such a scheme may be employed as to obtain the respective shift amounts of paired triangles between the fundus images and pick up one of them that has the highest frequency of appearance in overlapping processing etc.

Although the present embodiment has employed processing to overlap two fundus images, the present invention is not limited thereto. The scheme to be employed only needs to overlap the first and second fundus images or may process at least three fundus images. For example, in the case of merging a plurality of fundus images into a panoramic image, the aforesaid matching is performed so that one of the fundus images, that has a feature with the highest relevance ratio with the others, may provide a center of the panoramic image. It is thus possible to determine a center image even if the identification information etc. of the fundus images do not contain the position information of the fixation lamp.

Although in the above description, a scheme has been employed to process fundus images picked up with the same device such as a fundus camera, the present invention is not limited thereto. The scheme to be employed only needs to overlap a plurality of fundus images or extract blood vessel information. Such a scheme may be employed as to overlap a fundus image picked up with a fundus camera and that picked up with a scanning laser eye speculum each other. In such a case, such processing may be performed as to uniform the scale factors etc. of the fundus images and then extract blood vessel shapes from the respective fundus images and overlap the fundus images precisely. An allowable range employed in this case may be set in accordance with the type of the image pickup device employed.

Although the present embodiment has employed a scheme of acquiring a panoramic fundus image from a plurality of fundus images and integrating blood vessel shapes across those multiple fundus images to acquire fundus blood vessel information, the scheme only needs to acquire either one of them.

Further, the present invention is not limited to a scheme of acquiring a panoramic fundus image and/or fundus blood vessel information. The scheme only needs to be capable of aligning a plurality of fundus images or may align the fundus images of the same examinee's eye picked up at different dates and times. For example, such a scheme may be possible to extract a similar specific site in a second one of fundus images of almost the same site at different dates and times corresponding to a specific site (blood vessel or affected area) specified in a first one of these fundus images. In this case, the fundus blood vessel information is used to manage a specified position in the fundus image.

The above description is given by way of example, and not limitation. Given the above disclosure, one skilled in the art could devise variations that are within the scope and spirit of the invention disclosed herein, including various ways of using the disclosed image processing on other types of biological tissue, whether human or otherwise. Further, the various features of the embodiments disclosed herein can be used alone, or in varying combinations with each other and are not intended to be limited to the specific combination described herein. Thus, the scope of the claims is not limited by the illustrated embodiments.

What is claimed is:

1. A fundus information processing apparatus for processing a first fundus image and a second fundus image acquired with a fundus imaging device and acquiring at least one of a panoramic fundus image and fundus blood vessel information, the apparatus comprising:
a blood vessel extraction unit that extracts blood vessel shapes from the first and second fundus images, the unit extracting branching points of the blood vessel of the blood vessel shape through image processing;
a line segment information acquisition unit that acquires, in each of the first and second fundus images, information of a plurality of line segments obtained by interconnecting two predetermined branching points by using the plurality of branching points extracted by the blood vessel extraction unit;
a line segment identification unit that identifies at least two of the line segments common to the first and second fundus images by using the line segment information obtained by the line segment information acquisition unit;
a branching point information calculation unit that calculates, in each of the first and second fundus images, branching point information that indicates a relative positional relationship between the branching points constituting at least two of the line segments identified by the line segment identification unit;
a comparison operation unit that performs comparison operation on first branching point information of the first fundus image and second branching point information of the second fundus image which are calculated by the branching point information calculation unit; and
an alignment processing unit that performs alignment processing on the first and second fundus images based on a result of comparison by the comparison operation unit.

2. The fundus information processing apparatus according to claim 1, wherein the line segment information contains a length of the line segment and a formed angle.

3. The fundus information processing apparatus according to claim 1, wherein the branching point information calculation unit calculates information of a triangle formed by three branching points of the identified two line segments, in each of the first and second fundus images.

4. The fundus information processing apparatus according to claim 3, wherein the information of the triangle contains the respective lengths of two sides of the formed triangle and the angle formed by the two sides.

5. The fundus information processing apparatus according to claim 1, further comprising an overlapping unit that overlaps the first and second fundus images based on a result of the alignment by the alignment processing unit, thereby obtaining a panoramic fundus image.

6. The fundus information processing apparatus according to claim 1 further comprising a fundus blood vessel information calculation unit that calculates fundus blood vessel information which links the blood vessel shape of the first fundus image and the blood vessel shape of the second fundus image based on the result of the alignment by the alignment processing unit.

7. The fundus information processing apparatus according to claim 6, further comprising a blood vessel feature extraction unit that calculates any one of the number of blood vessel branches, the blood vessel branching shape, the length of the blood vessel between the branching points, and a width of the blood vessel from the fundus blood vessel information calculated by the fundus blood vessel information calculation unit.

8. The fundus information processing apparatus according to claim 1 to further comprising a boundary processing unit that, by using the alignment processing unit, sets up a boundary region at a location where the first and second fundus images overlap each other, performs comparison operation on the respective blood vessel shapes of the first and second fundus images, and aligns the first and second fundus images based on the result of the comparison operation.

9. The fundus information processing apparatus according to claim 1, further comprising a correction unit that corrects distortion in the first and second fundus images.

10. A fundus information processing method to process a first fundus image and a second fundus image acquired with a fundus imaging device to generate a panoramic fundus image and fundus blood vessel information comprising the following steps:
a blood vessel extraction step of extracting blood vessel shapes from the first and second fundus images, the step extracting branching points of the blood vessel of the blood vessel shape through image processing;
a line segment information acquisition step of acquiring, in each of the first and second fundus images, information of a plurality of line segments obtained by interconnecting two predetermined branching points by using the plurality of branching points extracted at the blood vessel extraction step;
a line segment identification step of identifying at least two of the line segments common to the first and second fundus images by using the line segment information obtained at the line segment information acquisition step;
a branching point information calculation step of calculating, in each of the first and second fundus images, branching point information that indicates a relative positional relationship between the branching points constituting at least two of the line segments identified at the line segment identification step;
a comparison operation step of performing comparison operation on first branching point information of the first fundus image and second branching point information of the second fundus image which are calculated at the branching point information calculation step; and
an alignment processing step of performing alignment processing on the first and second fundus images based on a result of the comparison operation at the comparison operation step.

11. A method of generating an image of biological tissue comprising:
generating a first image of a first region of tissue;
generating a second image a second region of tissue, wherein said second region includes at least a portion of the first region of tissue;
identifying blood vessels visible within the first and second images;
identifying the branch points of the identified blood vessels;
generating at least two line segments in said first and second images, said line segments representing an interconnection between branch points of identified blood vessels;
comparing line segments of said first and second images to identify at least two common line segments of said first and second images;
aligning the first and second images using the common line segments; and
generating a combined image.

12. The method of generating an image of biological tissue of claim 11 wherein said first and second steps of generating an image is completed using a digital camera to create digital images.

13. The method of generating an image of biological tissue of claim 11 wherein said first and second steps of generating first is completed using a fundus camera to create digital images.

14. The method of generating an image of biological tissue of claim 12 wherein said digital images are stored in a computer memory.

15. The method of generating an image of biological tissue of claim 14 wherein the step of identifying blood vessels within said first and second images is completed by extracting blood vessel shapes though image processing of the stored digital images.

16. The method of generating an image of biological tissue of claim 11 wherein the step of generating at least two line segments includes generation of information containing the length of the line and formed angle of the line.

17. The method of generating an image of biological tissue of claim 11 further comprising the step of calculating blood vessel branching point information to indicate relative positional relationship between branching points.

18. The method of generating an image of biological tissue of claim 17 wherein the calculating step uses information of a triangle formed by three branching points of at least two identified line segments.

19. The method of generating an image of biological tissue of claim 12 comprising the further step of image processing said digital images to remove image distortion.

20. The method of generating an image of biological tissue of claim 12 comprising the further step of image processing said digital images to remove image noise.

21. The method of generating an image of biological tissue of 11 further comprising the step of overlaying said first and second images with a grid to identify seed points of the image where blood vessels intersect the grid.

22. The method of generating an image of biological tissue of claim 21 further comprising the step of line scanning on a rotational axis about the seed point.

23. The method of generating an image of biological tissue of claim 22 wherein the line scanning distance is 10 pixels with the seed point as the center point of the line scan.
